(19) United States
(12) Patent Application Publication
(10) Pub. No.: US 2026/0048236 A1
(43) Pub. Date: Feb. 19, 2026

(54) FLEXIBLE CATHETER STEERING DEVICE

(71) Applicant: yoR Labs, Inc., Beaverton, OR (US)

(72) Inventors: Spencer Theodore Slam, Portland, OR (US); Dakota Pellegrino, Hillsboro, OR (US); Anshumali Roy, Portland, OR (US)

(21) Appl. No.: 19/126,518

(22) PCT Filed: Nov. 6, 2023
(86) PCT No.: PCT/US2023/078858

§ 371 Date: May 1, 2025

Related U.S. Application Data
(60) Provisional application No. 63/382,761, filed on Nov. 8, 2022.

(51) Int. Cl. A61M 25/01; A61M 25/00

(52) U.S. Cl. CPC A61M 25/0136; A61M 25/0054; A61M 25/0147; A61M 25/0155

(57) ABSTRACT

A catheter steering system wherein the user interface device is a flexible shaft that can be manipulated by hand. The motion imparted on the flexible steering device is reflected in the motion of the catheter tip through an arrangement of pull wires or other mechanisms. Through this arrangement a more intuitive control of the catheter tip is obtained. In an example, a catheter control device includes a catheter port configured to receive the proximal end of a catheter that includes one or more pull wires for attachment to a swashplate, a swashplate, a steering shaft configured to bend in response to a force being applied to the steering shaft; and a mechanism configured to translate the bend of the steering shaft to movement of the swashplate to correspondingly move the distal tip of a catheter coupled to the swashplate.

### INCORPORATION BY REFERENCE TO ANY PRIORITY APPLICATIONS

Any and all applications for which a foreign or domestic priority claim is identified in the Application Data Sheet as filed with the present application are hereby incorporated by reference under 37 CFR 1.57. This disclosure is related to PCT/US2023/078858, filed on Nov. 6, 2023, and U.S. Provisional No. 63/382,761, filed on Nov. 8, 2022, both of which are incorporated by reference herein in their entirety.

### TECHNICAL FIELD

The invention relates to catheter systems for controlling movement of a distal end of a catheter.

### BACKGROUND

Control and movement (e.g., bending) of a distal end of a catheter is typically done by rotational movement of a dial or another type of actuator on a catheter handle coupled to the catheter. However, the movement of the actuator often does not intuitively correspond to the bending movement of the catheter distal tip. For example, a catheter handle may have or more dials arranged around the longitudinal axis of the handle and perpendicular to the longitudinal axis, and rotation of a dial moves the catheter distal tip in a posterior or anterior direction, or a left or right direction. Accordingly, it would be advantageous for a catheter steering mechanism to provide control of the movement of the catheter distal tip in a more intuitive way manner.

### SUMMARY

Certain aspects of this invention are defined by the independent claims. The dependent claims concern optional features of some embodiments of the invention. The systems, methods, and devices described herein each have several aspects, no single one of which is solely responsible for its desirable attributes. Without limiting the scope of this disclosure, several non-limiting features will now be discussed briefly.

In one aspect of the system and methods disclosed herein, a catheter control device is described. The catheter control device includes a catheter port, a flexible steering shaft, one or more pulleys, and one or more steering wires. The catheter port is configured to receive the proximal end of a catheter, wherein the catheter comprises one or more pull wires. The flexible steering shaft are configured to bend in response to a force being applied to the flexible steering shaft. The one or more steering wires are disposed along the flexible steering shaft, routed through the one or more pulleys, and connected to the one or more pull wires of the catheter via the catheter port. In response to the force being applied to the flexible steering shaft, the one or more steering wires and the one or more pulleys are configured to transfer the force to the one or more pull wires of the catheter such that a distal end of the catheter moves in the same direction as the flexible steering shaft.

The above and other aspects have various embodiments. For example, in some embodiments, the flexible steering shaft includes one or more of: an elastomer, a metallic spring, and a flexible metal tubing. In some embodiments, the flexible steering shaft includes two or more rigid members wherein the two or more rigid members are flexibly connected. In some embodiments, the catheter port is further configured to receive a swashplate of the catheter and connect the one or more pull wires of the catheter to the one or more steering wires via the swashplate of the catheter.

In another aspect, a catheter control device is described that includes a catheter port, a flexible steering shaft, a threaded member, a gear, and one or more steering wires. The catheter port is configured to receive the proximal end of a catheter, wherein the catheter comprises one or more pull wires. The flexible steering shaft is configured to bend in response to a force being applied to the flexible steering shaft. The threaded member is connected to the one or more pull wires via the catheter port. The gear is disposed along the threaded member such that the teeth of the gear interfaces with the threads of the threaded member. The one or more steering wires are disposed along the flexible steering shaft and connected to the gear. The one or more steering wires, the gear, and the threaded member are configured to, in response to the force being applied to the flexible steering shaft, transfer the force to the one or more pull wires of the catheter such that a distal end of the catheter moves in the same direction as the flexible steering shaft.

The above and other aspects have various embodiments. For example, in some embodiments, the flexible steering shaft includes one or more of: an elastomer, a metallic spring, and a flexible metal tubing. In some embodiments, the flexible steering shaft includes two or more rigid members, and wherein the two or more rigid members are flexibly connected. In some embodiments, the catheter port is further configured to receive a swashplate of the catheter and connect the one or more pull wires of the catheter to the threaded member via the swashplate of the catheter.

In another aspect, a catheter control device is disclosed that includes a catheter port, a flexible steering shaft, and a hydraulic actuator. The catheter port is configured to receive the proximal end of a catheter, wherein the catheter comprises one or more pull wires. The flexible steering shaft is configured to bend in response to a force being applied to the flexible steering shaft. The hydraulic actuator includes a cylinder, a piston, a first pipe, and a second pipe. The piston is connected to the one or more pull wires via the catheter port. The first pipe originates at a first end of the cylinder and terminates at a distal end of the flexible steering shaft. The second pipe originates at a second end of the cylinder and terminates at the distal end of the flexible steering shaft. The second end of the cylinder is opposite the first end of the cylinder. The hydraulic actuator is configured to, in response to the force being applied to the flexible steering shaft, transfer the force to the one or more pull wires of the catheter such that a distal end of the catheter moves in the same direction as the flexible steering shaft.

The above and other aspects have various embodiments. For example, in some embodiments, the flexible steering shaft comprises one or more of: an elastomer, a metallic spring, and a flexible metal tubing. In some embodiments, the flexible steering shaft comprises a two or more rigid members, and wherein the two or more rigid members are flexibly connected. In some embodiments, the catheter port is further configured to receive a swashplate of the catheter and connect the piston to the one or more steering wires via the swashplate of the catheter.

In another aspect, a catheter control device is disclosed that includes a catheter port, a swashplate, a steering shaft, and a mechanism. The catheter port is configured to receive the proximal end of a catheter that includes one or more pull wires for attachment to a swashplate. The steering shaft is configured to bend in response to a force being applied to the steering shaft. The mechanism is configured to translate the bend of the steering shaft to movement of the swashplate to correspondingly move the distal tip of a catheter coupled to the swashplate.

The above and other aspects have various embodiments. For example, in some embodiments, the mechanism includes a wire and at least one pulley. In some embodiments, the mechanism includes at least one hydraulic line and hydraulic fluid. In some embodiments, the mechanism a gear assembly. In some embodiments, the mechanism is configured to move the swashplate to correspondingly move a distal tip of a catheter coupled to the swashplate in an anterior and posterior direction. In some embodiments, the mechanism is further configured to move the swashplate to correspondingly move a distal tip of a catheter coupled to the swashplate in a left and right direction. In some embodiments, the mechanism is configured to move the swashplate to correspondingly move a distal tip of a catheter coupled to the swashplate in a left and right direction.

### BRIEF DESCRIPTION OF THE DRAWINGS

FIG. 1 is a lateral cutaway view of an example catheter system that employs a wire and pulley system that can move a plate when the handle is moved to correspondingly move a distal tip of a catheter;

FIG. 2 is a lateral cutaway view of an example of a catheter steering device of FIG. 1 showing an example of its use;

FIG. 3 is a lateral cutaway view of an example of a catheter steering device that includes a gear system to move a plate when the handle is moved to correspondingly moves a distal tip of a catheter;

FIG. 4 is an example of a catheter steering device with a steering shaft that is moved by a user's hand, the steering shaft comprising multiple rigid members flexibly connected by ball joints; and

FIG. 5 is a lateral cutaway view of another example catheter steering device that includes a hydraulic system to move a plate when the handle is moved to correspondingly move a distal tip of a catheter.

### DETAILED DESCRIPTION

Embodiments disclosed herein relate to a method of controlling, or steering, a catheter with a more intuitive control. Embodiments of the steering device provide an interface which mimics the form and motion of the bending of a flexible catheter tip (i.e., the distal tip of the catheter). In some embodiments, the operator may bend the handle (or steering shaft) to the desired direction and angle to achieve a similar motion at the distal tip of the catheter. Mechanical gain may be a one-to-one mapping from steering shaft angulation to catheter tip angulation, or may be an alternative ratio (e.g., reducing the gain to allow for smaller catheter tip movements). In some embodiments, the flexible steering shaft may be combined with other controls, for example, rotating dial or knob controls, to allow for fine adjustment of the catheter tip position.

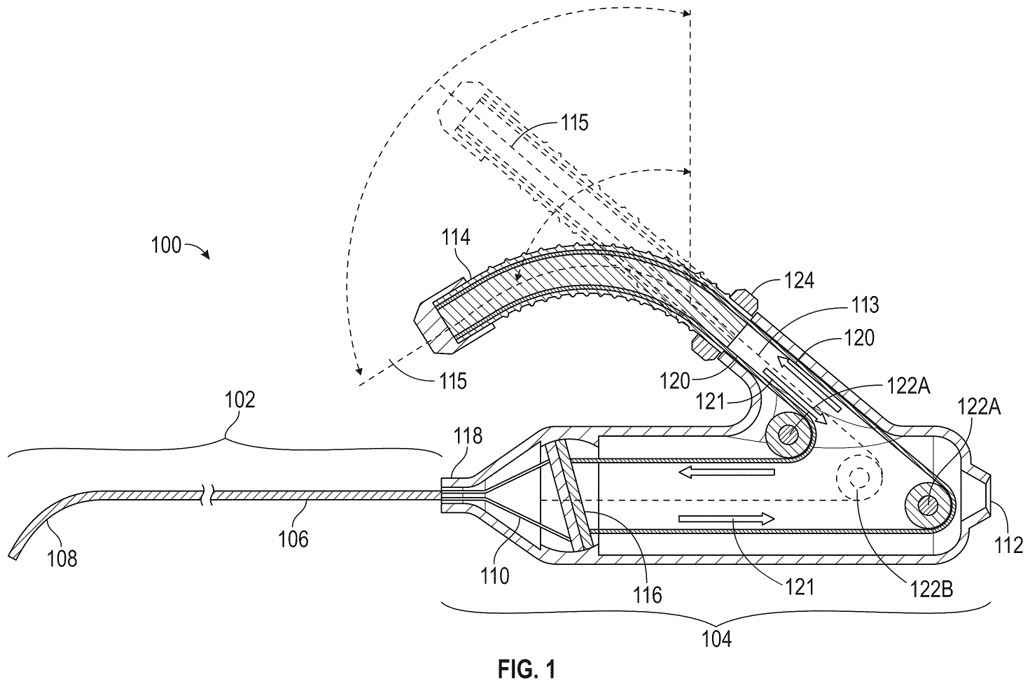

FIG. 1 is an example embodiment of a catheter system 100. The catheter system 100 includes a catheter 102 and a catheter steering device 104. The catheter has a proximal end 106 and a distal end 108. The catheter 102 may also include one or more pull wires 110 to articulate the distal end 108 of the catheter in a multitude of directions. For example, the one or more pull wires can be used to move the distal end 108 of the catheter 102 downward as shown in FIG. 1. Using one or more pull wires, the distal end 108 of the catheter may be moved up, down, left, right, or any combination thereof. In some embodiments, the entire catheter 102 may be made of a flexible material. Various embodiments of the catheter 102 can be structured in a variety of configurations to navigate intracorporeal spaces and/or perform certain procedures. The catheter 102 may have multiple articulation regions, including cardiac regions. The catheter 102 may be any type of catheter. For example, in some embodiments, the catheter may be an intracardiac echocardiography (ICE) catheter, an intravascular ultrasound (IVUS) catheter, a radiofrequency (RF) ablation catheter, or a fractional flow reserve (FFR) catheter modality. In some embodiments, the user may indicate, set, or add an articulation region through a mechanism on the catheter steering device.

The catheter steering device 104 includes a body 112 having a centerline 113, a steering shaft 114 having a centerline 115, a swash plate 116, a catheter receiving port 118 and one or more steering wires 120. In some embodiments, such as the one shown in FIG. 1, the catheter steering device 104 includes one or more pulleys 122. The body 112 can form a housing to contain the swash plate 116 and the one or more steering wires 120. The body can also form the catheter receiving port 118. The steering shaft 114 may be a bendable member. In some embodiments, the steering shaft 114 may be composed of an elastomer, metallic spring, or flexible metal tubing, or a combination thereof. The steering shaft 114 may include an internal component that is surrounded by an outer layer. For example, the internal component may be a flexible elastomer, and the outer layer may be a sterilizable coating. In some embodiments, the steering shaft 114 may be a rigid member that moves forward, backward, left, right, or any appropriate combination thereof. For example, the steering shaft 114 may be a rigid joystick that is attached at one end to a ball and socket joint and configured to around the joint to dictate different movements. In some embodiments, the steering shaft 114 may be deflected relative to an initial or normal position. The initial or normal position can correspond to a position when the centerline 115 of the steering shaft 114 is colinear with a centerline 113 of the body 112. The steering shaft 114 can be deflected from the initial or normal position to manipulate the swash plate 116 via the one or more steering pull wires 120. In some embodiments, the steering shaft 114 can be deflected proximally or distally from the body 112 as illustrated in FIG. 1. In some embodiments, the steering shaft 114 can be deflected along a plane coplanar with the centerline 113 of the body 112. In any of the disclosed steering mechanisms disclosed herein, in some embodiments, the steering device can be configured to move the distal tip 108 in a plane (e.g., up and down, or left and right). In some embodiments, the steering device 104 can be configured to move the distal tip 108 in two planes (e.g., up and down, and left and right). In some embodiments, any of the components that translate movement of the steering shaft 114 to movement of the swash plate 116 may be referred to as the steering mechanism. For example, the steering mechanism may include one or more of the following: steering wires, steering pull wires, pulleys, a steering gear, a steering wire-gear interface, a hydraulic actuator, a cylinder, and/or a piston.

The steering shaft 114 may include internal passageways or channels that house the one or more steering wires 120. The internal passageways may terminate at a distal end of the steering shaft 114, and the one or more steering wires 120 may also be attached at the distal end of the steering shaft 114. In some embodiments, the internal passageways may terminate before the distal end of the steering shaft 114. The internal passageways may be circumferentially disposed around the centerline 115 of the steering shaft 114 such that the passageways are evenly spaced around the centerline 115 relative to one another. The passageways may be placed at various distances, i.e., radii, from the centerline 115. For example, as shown in FIG. 1, the passageways, and by proxy the one or more steering pull wires 120, may be placed at an outer diameter of the steering shaft 114. In some embodiments, the one or more steering wires 120 may be arranged at different distances from the centerline 115. The one or more steering wires 120 may also be unevenly spaced around the centerline 115. The one or more steering wires 120 may be disposed around the centerline 115 of the steering shaft based on a desired movement of the distal end 108 of the catheter 102. In some embodiments, the one or more steering wires 120 may be coplanar about a central plane of the catheter system 100 as shown in FIG. 1. In some embodiments, the one or more steering wires 120 may be coplanar about a traverse plane through the catheter system 100 as illustrated by the dotted line extending over the pulley 122B.

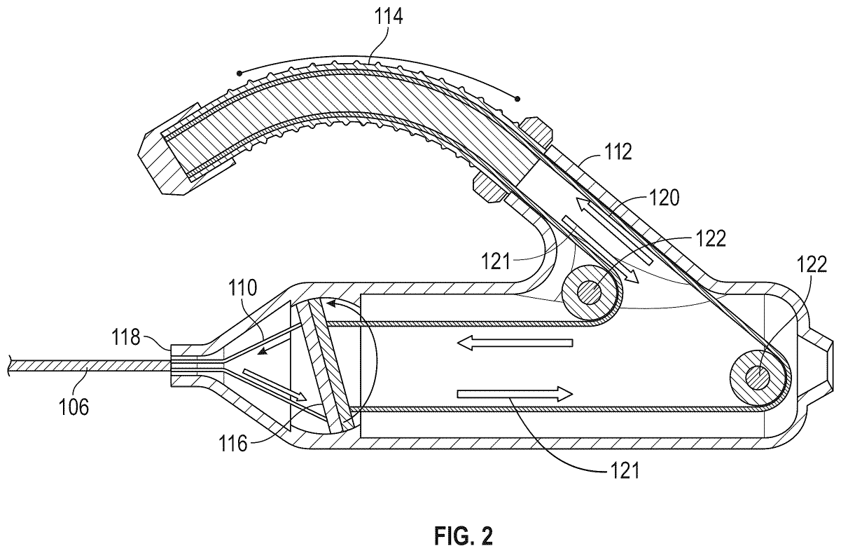

The one or more steering wires 120 may exit the internal passageways at a proximal end of the steering shaft 114, pass through the body 112, and connect to the swashplate 116. The one or more steering wires 120 may pass through one or more pulleys 122 or systems of pulleys within the body 112 before reaching the swashplate 116. For example, the catheter steering device 104 may include 2 steering wires 120, and the 2 steering wires may each pass through a corresponding pulley 122. The path of the one or more steering wires 120 through the pulleys 122 may determine a movement ratio of the steering shaft 114 to the distal end 108 of the catheter 102. The movement ratio for the embodiment illustrated in FIG. 1, or any of the disclosed embodiments in FIGS. 2-5. may be a particular value, e.g., 1:1, 2:1, 3:1, 4:1, 5:1, 6:1, 7:1, 8:1, 9:1, or 10:1 or any ratio therebetween. In some embodiments, the movement ratio of the steering shaft 114 to the distal end 108 of the catheter 102 is greater than 10:1, for example, 20:1, 30:1, 40:1, 50:1, 60:1, 70:1, 80:1, 90:1, or 100:1 or any ration therebetween, In some embodiments the movement ratio of the steering shaft 114 to the distal end 108 of the catheter 102 is greater than 100:1, for example, up to 1,000:1, or more. In some embodiments, the steering wires 120 may pass through a series of pulleys that increase or decrease the movement ratio. Deflecting the steering shaft 114 can tension at least one of the one or more steering wires 120 to pull the at least one of the one or more steering wires 120 toward the distal end of the steering shaft 114. The at least one of the one or more steering wires 120 tensioned by a deflection of the steering shaft 114 can pull on the swash plate 116 to pivot the swash plate 116. The pivoting of the swash plate 116 can pull on at least another of the one or more steering wires 120 to tension the other one or more steering wires 120 toward the swash plate 116. For example, as shown in FIG. 1, the steering shaft 114 can be deflected toward the body 112 to pivot the swash plate 116 by moving the one or more steering wires 120 in the directions as illustrated by the arrows 121.

In some embodiments, the catheter device 104 may include a switch or lever that engages or disengages additional pulleys to modulate (or change) the movement ratio. For example, the catheter steering device 104 may include a “fine motion” switch that engages an additional pulley to increase the movement ratio. The movement ratio may be determined by the dimensions of the steering shaft 114, such as the length. In some embodiments, the dimensions of the steering shaft 114 may be modular. For example, an extender may be placed on the distal end of the steering shaft 114 to increase the length of the steering shaft 114 and alter the movement ratio between the steering shaft 114 and the distal end 108 of the catheter 102. In some embodiments, the one or more steering wires 120 may be anchored to the body 110, and one of the pulleys 122 may be connected to the swashplate 116 such that a movement of the pulley attached to the swashplate 116 moves the swashplate 116.

The swashplate 116 may be connected to the one or more catheter pull wires 110 via the catheter receiving port 118. Examples of a swashplate 116 are described in more detail in PCT Application PCT/US23/73371, the entirety of which is hereby incorporated by reference. The one or more catheter pull wires 110 may pass through the catheter receiving port 116 and connect directly to the swashplate 114. In some embodiments, the one or more pull wires may terminate at the catheter receiving port 116. The catheter receiving port 116 may include a wire interface for transferring motion from the swashplate 116. The swashplate 116 allows the catheter 102 to be rotated about its own axis while maintaining a particular bend angle. In some embodiments, the steering shaft 114 may be configured to dictate a rotation of the catheter 102. For example, the steering shaft 114 may be rotatably attached to the catheter steering device 104 and configured such that the catheter 102 is rotated as the steering shaft 114 is rotated by a user. In some embodiments, the catheter steering device 104 may include a switch, level, or other mechanism to rotate the catheter 102.

In some embodiments, the catheter steering device 104 includes a locking collar 124. The locking collar 124 may be engaged to prevent movement of the steering shaft 114 and/or the one or more steering wires 120. For example, the locking collar 124 may clamp down on the steering wires 120 to prevent them from moving. In some embodiments, the locking collar 124 may include a cam mechanism that causes the locking collar 124 to cinch down on the one or more steering wires 120. In some embodiments, the locking collar 124 may be a threaded member that engages with a corresponding threaded member on the steering shaft 116 or the body 110, such that the locking collar 124 cinches down on the one or more steering wires 120 when the locking collar 124 is engaged with the treaded member of the steering shaft.

FIG. 2 is a lateral cutaway view of the catheter system 100 in use. When the catheter 102 is inserted into a patient, a user may move the steering shaft 114 to intuitively control the movement of the distal end 108 of the catheter 102. For example, the catheter 102 may be inserted into the femoral artery of a patient to analyze the calcification of the patient's aortic valve. In order to reach the heart, the distal end 108 of the catheter 102 may be turned or flexed as it passes through the patient's vasculature. As the catheter 102 is moved through the patient, a user may articulate the steering shaft 114 in a direction that the user wishes to move the distal end 108 of the catheter 102. For example, as shown in FIG. 2, the user may want to articulate the distal end 108 of the catheter 102 to downward and may move the steering member 114 downward. The steering shaft 114 may bend or flex down as the user “steers” the distal end 108 of the catheter 102.

As the steering shaft 114 bends, the one or more steering wires 120 move along with the steering shaft 114 within the passageways as the steering shaft 114 bends. In turn, the one or more steering wires 120 moves, or rotates, the swashplate 116. The swashplate 116 transfers, or couples, the motion to the one or more catheter pull wires 110, which causes the distal end 108 of the catheter 102 to move in the same direction as the steering shaft 114. In some embodiments, components of the catheter steering device 104 may limit the motion of the distal end 108 of the catheter 102 to prevent damage to the catheter 102. For example, the swashplate 116 may be placed in a housing that prevents the swashplate from being moved too far one direction. The steering shaft 114 may also have a limited range of motion according to some embodiments. In some embodiments, the steering shaft 114, the swashplate 116, the one or more steering wires 120, the one or more pulleys 122, may be arranged such that the distal end 108 of the catheter 102 mirrors the movement of the steering shaft 114. The steering shaft 114, the swashplate 116, the one or more steering wires 120, the one or more pulleys 122, may be arranged such that an inverted motion of the steering shaft 114 is transferred to the distal end of the catheter 102. For example, if the user bends the steering shaft down 114, an upward motion may be transferred to the distal end 108 of the catheter 102. In some embodiments, the catheter steering device 104 may include a switch, level, or other mechanism to switch between a “mirror mode” and an “inverted mode.”

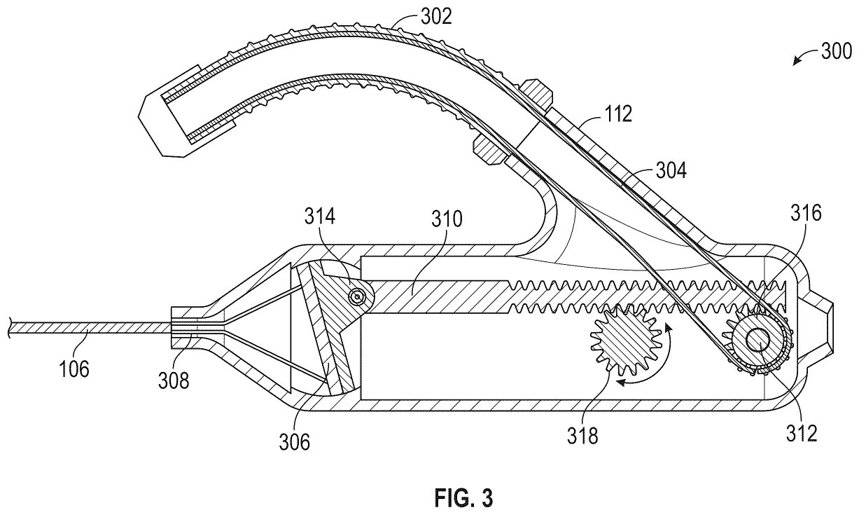

FIG. 3 is a lateral cutaway view of an example embodiment of a catheter steering device 300. Similar to the catheter steering device 104, the catheter steering device 300 includes a steering shaft 302, one or more steering wires 304, a swashplate 306, and one or more catheter pull wires 308. In some embodiments, the catheter steering device 300 includes a threaded member 310 and a steering gear 312. The threaded member 310 may include engagement members (threads or teeth) that extend longitudinally along the body of the threaded member 310. In some embodiments, the threaded member 310 may be a rack member that includes teeth placed longitudinally along the body of the threaded member 310. In some embodiments, the threaded member 310 may be a lead screw that includes threads that extend longitudinally along the body of the threaded member 310. Other suitable engagement members may also be used, according to other embodiments.

The threaded member 310 may be attached to the swashplate 306 at a joint 314 such as a knuckle joint. Other joints, such as a ball joint may also be employed. The steering gear 312 may be placed such that the teeth of the steering gear contact the threads of the threaded member 310. The one or more steering wires 304 may be attached to the steering gear 312 at a steering wire-gear interface 316. For example, the interface 316 may be an annular protrusion that receives the one or more steering wires 304. The protrusion may also include a slot where the one or more steering wires may be fixed to the steering gear 312.

In some embodiments, the catheter steering device 300 includes two or more steering gears arranged such that a movement ratio between the steering shaft 302 and a distal end of a catheter is a particular value, for example, 1:1, 2:1, 3:1, 4:1, 5:1, 10:1, 100:1, or 1,000:1, or any ratio in-between these ratios. The gear ratio may dictate the movement ratio. In some embodiments, the catheter steering device 300 may include a switch, lever, or other engagement mechanism that can add or subtract gears from the drive train between the threaded member 310 and the steering gear 312.

The catheter steering device 300 may also include another threaded member and steering gear configured to move the swashplate in another plane of motion. For example, the threaded member 310 and the steering gear 312 may dictate the motion of the swashplate 306 in a particular plane, such as a vertical (up-down) plane. The catheter steering device 300 may include another threaded member and steering gear system (a second gear steering system) that controls the movement of the swashplate 306 in a horizontal (left-right) plane. The second gear steering system may be connected to the steering shaft 302 or to a second steering shaft.

The catheter steering system 300 may also include a fine control gear 318, another gear member paired to the threaded member. The fine control gear 318 may be connected to an external knob on the exterior of the catheter steering device 300. For example, the fine control gear 318 may be connected to the external knob via a shaft. The external knob and the fine control gear 318 may also be used to control the movement of a distal end of a catheter. The fine control gear 318 may have a higher gear ratio than the steering gear 312. In some embodiments, the fine control gear 318 may be two or more gears. For example, a first fine control gear may be attached to the external knob, and a second fine control gear may be placed in a drivetrain between the first fine control gear and the threaded member 310.

As the user bends the steering shaft 302 to steer a distal end of a catheter, the one or more steering wires 304 may be pulled along passageways with the steering shaft 302 as discussed above in greater detail. The pulling of the one or more steering wires 304 in turn causes the steering gear 312 to turn in one direction. As the steering gear 312 turns, the teeth of the steering gear 312 interact with the threaded member 310, moving the threaded member 310 forward or backward along a path. The movement of the threaded member 310 moves, or rotates, the swashplate 306 at the joint 314. As the swashplate 306 rotates, force is applied to the one or more catheter pull wires 308 such that the distal end of a catheter is moved in the same direction as the steering shaft 302. For example, if the steering shaft 302 was bent down by the user, the distal end of the catheter is steered downwards. The one or more steering wires 304, the swash plate 306, the threaded member 310 and the steering gear 312 can be have any angular position within the body 112. For example, in some embodiments, the one or more steering wires 304, the swash plate 306, the threaded member 310 and the steering gear 312 can be rotated 90 degrees relative to the embodiments shown in FIG. 3.

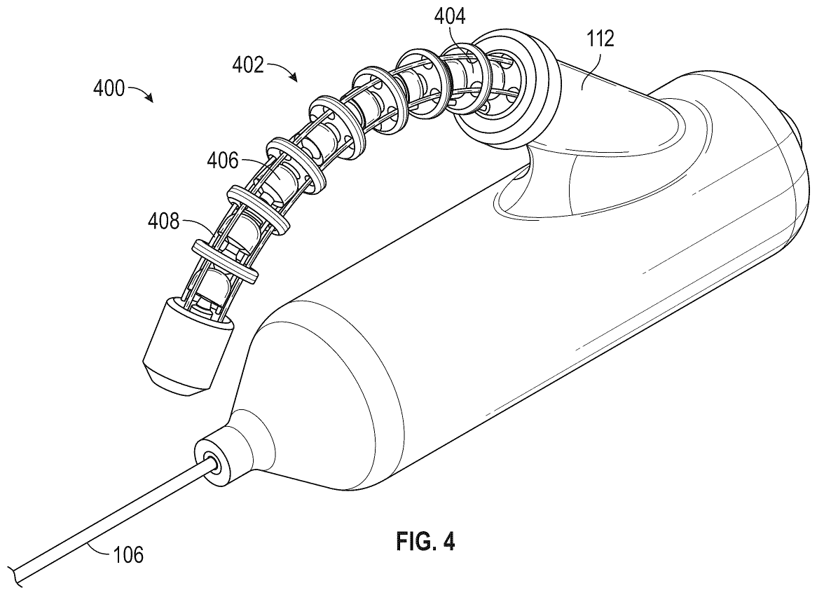

FIG. 4 shows an example embodiment of a catheter steering device 400 with a steering shaft 402. The steering shaft 402 is comprised of multiple rigid members 404 that are flexibly connected by ball joints 406. The one or more steering wires 408 may be threaded through the rigid members 404 and attached at a distal end of the steering shaft 402. In some embodiments, the one or more steering wires 408 may terminate at different locations along the steering shaft 502 and provide multiple bending regions within the steering shaft 502 and a distal end of the catheter. The one or more steering wires 408 may provide tension to the steering shaft 402 such that the steering shaft 402 remains static when not in use. In some embodiments, the friction between the rigid members 404 may prevent the steering shaft from moving while not in use. For example, the interior surface of the ball joints 406 may be coated to increase the friction between the rigid members 404 to prevent slipping. In some embodiments, the catheter steering device 400 may include a locking collar, similar to the locking collar 124, that locks the steering shaft 402 in place and prevents unwanted movement.

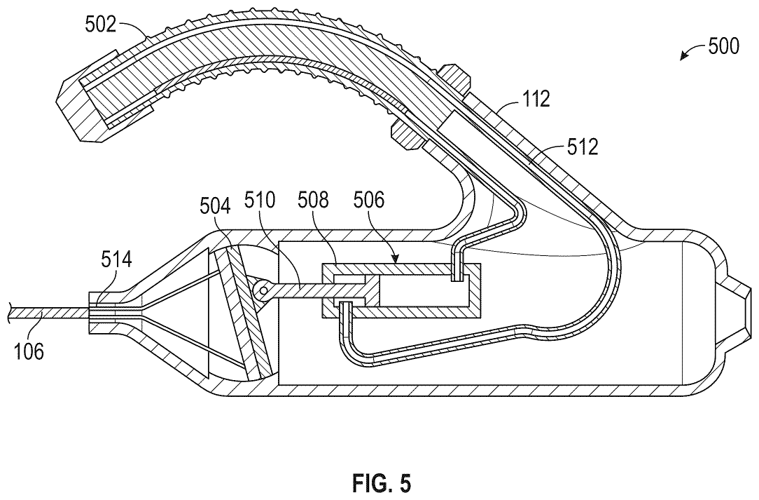

FIG. 5 is a cutaway lateral view of an example catheter steering device 500 that employs a hydraulic system to transfer motion between the steering shaft 502 and the swash plate 504, which may be configured similar to the steering shaft 114 and the swashplate 116 of FIG. 1. The catheter steering device 500 may include a hydraulic actuator 506. The hydraulic actuator may have a cylinder 508 that partially houses a piston 510 configured to translate pressure within the hydraulic actuator 506 into a linear motion. The piston 510 may extend from one end of the cylinder 508 and may be attached to the swashplate 504 at a joint, such as a knuckle joint.

The catheter steering device 500 may also include two or more pipes 512 that extend from opposite ends of the cylinder 508 and terminate at a distal end of the steering shaft 502. Similar to the passageways discussed above in conjunction with FIGS. 1 and 2, the two or more pipes 512 may be opposed to one another across the centerline of the steering shaft 502. For example, the first pipe may be placed on a top side of the steering member 502, and the second pipe may be diametrically opposed to the first pipe and placed on the bottom of the steering member 502. The cylinder 508 and the pipes 512 may be filled with a hydraulic fluid. The amount of hydraulic fluid placed within the hydraulic actuator 506, and the viscosity of the fluid may determine a movement ratio of the steering shaft 502 to the distal end of the catheter. The dimensions of the cylinder 508, piston 510, and the one or more pipes 512 may also affect the movement ratio of the steering member to the distal end of the catheter.

When the catheter steering device 500 is in operation, a user may dictate a movement of the distal end of a catheter by moving the steering shaft 502 in a particular direction. For example, the user may bend the steering shaft 502 down. As the steering shaft 502 is bent, the hydraulic fluid within the one or more pipes 512 in the steering shaft 502 is compressed. The pressure in the one or more pipes 512 presses on the piston 510, causing the piston to move 510 and transferring the motion to the swashplate 504. In turn, the swashplate moves, or rotates, causing the one or more catheter pull wires 514 to articulate the distal end of the catheter in the same direction that the steering shaft was moved. For example, if the steering shaft 502 is bent down, fluid in the one or more pipes 512 at the bottom of the steering member is compressed, putting pressure on the left side of the piston 508 and causing the piston to move to the right. The piston 508 transfers this motion to the swashplate 506, causing the distal end of the catheter to be moved in a downward direction.

In some embodiments, the hydraulic actuator 506 may control the movement of the distal end of a catheter in a particular place of motion, such as a vertical plane. In some embodiments, the catheter steering device 500 may include another hydraulic actuator that is configured similar to the hydraulic actuator 506 but configured to control movement of the distal end of a catheter in another plane of motion, such as a horizontal plane. The swashplate 504, the piston 510, and the one or more pipes 512, can be have any angular position within the body 112. For example, in some embodiments, the swashplate 504, the piston 510, and the one or more pipes 512 can be rotated 90 degrees relative to the embodiments shown in FIG. 5.

### Example Embodiments

In a first example embodiment, the steering shaft is of a fully flexible construction similar to the design of a catheter shaft. The shaft can be composed of an elastomer, metallic spring, flexible metal tubing or a combination thereof. Internal to the shaft are passageways arrayed about the centerline. These passageways contain the steering pull wires, which may terminate at the distal end of the steering shaft. The angulation of the shaft lengthens or shortens the path length of the steering pull wires. This change in length (pull) is connected to the pull wires in the catheter.

The connection to the catheter pull wires may be a direct connection, routed through pulleys. Alternatively, the steering wires may be connected to a mechanism internal to the handle which in turn provides an interface to the catheter pull wires. The illustrated embodiment (as shown in FIGS. 1 and 2) includes a pivoting swashplate that would provide an interface to the catheter pull wires. This allows the operator to easily rotate the catheter about its own axis while maintaining a particular bend angle, thereby allowing the operator to steer the catheter imaging plane.

The operator of the device may wish to lock the steering shaft (and connected catheter) into a particular orientation in order to let go of the handle and perform other tasks. Position locking of the steering shaft can be achieved by clamping the pull wires at the base of the steering shaft. This can be accomplished through a cam mechanism in the locking collar, for example.

In an alternative embodiment that is similar to the embodiment shown in FIG. 3, the steering pull wires serve as the input to a gear train which in turn manipulates the catheter pull wires. This can allow for variations in the gain between steering handle input and catheter tip output. Additionally, the gear train can accept additional inputs to enable fine control of the catheter tip with a knob, for example.

In an alternative embodiment that is similar to the embodiment shown in FIG. 4, the flexible steering shaft is composed of a vertebrae-like arrangement of rigid links and joints with a number of pull wires routed through the series of links. Like the previous embodiments, angulation of the shaft changes the path length of the pull wires. The segmented flexible input device can also be used for the control of a catheter with multiple bending regions by terminating multiple sets of steering pull wires at different points along the length of the steering shaft. In this manner, more complex multi-bend operations can intuitively be performed.

In another example embodiment that is similar to the embodiment shown in FIG. 5, steering pull wires can be replaced by a hydraulic mechanism. Sealed cavities are arrayed about the steering shaft centerline, filled with incompressible fluid and connected to hydraulic actuators (single or double acting). As the steering shaft is bent, the sealed cavity on the inside of the bend is compressed, thereby pushing fluid into a hydraulic actuator, which in turn manipulates the catheter pull wires.

The foregoing description details certain embodiments of the systems, devices, and methods disclosed herein. It will be appreciated, however, that no matter how detailed the foregoing appears in text, the systems, devices, and methods can be practiced in many ways. As is also stated above, it should be noted that the use of particular terminology when describing certain features or aspects of the embodiments should not be taken to imply that the terminology is being re-defined herein to be restricted to including any specific characteristics of the features or aspects of the technology with which that terminology is associated.

Conditional language such as, among others, “can,” “could,” “might” or “may,” unless specifically stated otherwise, are otherwise understood within the context as used in general to convey that certain embodiments include, while other embodiments do not include, certain features, elements and/or steps. Thus, such conditional language is not generally intended to imply that features, elements and/or steps are in any way required for one or more embodiments or that one or more embodiments necessarily include logic for deciding, with or without user input or prompting, whether these features, elements and/or steps are included or are to be performed in any particular embodiment.

Headings are included herein for reference and to aid in locating various sections. These headings are not intended to limit the scope of the concepts described with respect thereto. Such concepts may have applicability throughout the entire specification.

Many variations and modifications may be made to the above-described embodiments, the elements of which are to be understood as being among other acceptable examples. All such modifications and variations are intended to be included herein within the scope of this disclosure. The foregoing description details certain embodiments. It will be appreciated, however, that no matter how detailed the foregoing appears in text, the systems and methods can be practiced in many ways. As is also stated above, it should be noted that the use of particular terminology when describing certain features or aspects of the systems and methods should not be taken to imply that the terminology is being re-defined herein to be restricted to including any specific characteristics of the features or aspects of the systems and methods with which that terminology is associated.

It will also be understood that, when a feature or element (for example, a structural feature or element) is referred to as being “connected”, “attached” or “coupled” to another feature or element, it may be directly connected, attached or coupled to the other feature or element or intervening features or elements may be present. In contrast, when a feature or element is referred to as being “directly connected”, “directly attached” or “directly coupled” to another feature or element, there may be no intervening features or elements present. Although described or shown with respect to one embodiment, the features and elements so described or shown may apply to other embodiments. It will also be appreciated by those of skill in the art that references to a structure or feature that is disposed “adjacent” another feature may have portions that overlap or underlie the adjacent feature.

Terminology used herein is for the purpose of describing particular embodiments and implementations only and is not intended to be limiting. For example, as used herein, the singular forms “a”, “an” and “the” may be intended to include the plural forms as well, unless the context clearly indicates otherwise. It will be further understood that the terms “comprises” and/or “comprising,” when used in this specification, specify the presence of stated features, steps, operations, processes, functions, elements, and/or components, but do not preclude the presence or addition of one or more other features, steps, operations, processes, functions, elements, components, and/or groups thereof. As used herein, the term “and/or” includes any and all combinations of one or more of the associated listed items and may be abbreviated as “/”.

In the descriptions above and in the claims, phrases such as “at least one of” or “one or more of” may occur followed by a conjunctive list of elements or features. The term “and/or” may also occur in a list of two or more elements or features. Unless otherwise implicitly or explicitly contradicted by the context in which it used, such a phrase is intended to mean any of the listed elements or features individually or any of the recited elements or features in combination with any of the other recited elements or features. For example, the phrases “at least one of A and B;” “one or more of A and B;” and “A and/or B” are each intended to mean “A alone, B alone, or A and B together.” A similar interpretation is also intended for lists including three or more items. For example, the phrases “at least one of A, B, and C;” “one or more of A, B, and C;” and “A, B, and/or C” are each intended to mean “A alone, B alone, C alone, A and B together, A and C together, B and C together, or A and B and C together.” Use of the term “based on,” above and in the claims is intended to mean, “based at least in part on,” such that an unrecited feature or element is also permissible.

Spatially relative terms, such as “forward”, “rearward”, “under”, “below”, “lower”, “over”, “upper” and the like, may be used herein for ease of description to describe one element or feature's relationship to another element(s) or feature(s) as illustrated in the figures. It will be understood that the spatially relative terms are intended to encompass different orientations of the device in use or operation in addition to the orientation depicted in the figures. For example, if a device in the figures is inverted, elements described as “under” or “beneath” other elements or features would then be oriented “over” the other elements or features due to the inverted state. Thus, the term “under” may encompass both an orientation of over and under, depending on the point of reference or orientation. The device may be otherwise oriented (rotated 90 degrees or at other orientations) and the spatially relative descriptors used herein interpreted accordingly. Similarly, the terms “upwardly”, “downwardly”, “vertical”, “horizontal” and the like may be used herein for the purpose of explanation only unless specifically indicated otherwise.

Although various illustrative embodiments have been disclosed, any of a number of changes may be made to various embodiments without departing from the teachings herein. For example, the order in which various described method steps are performed may be changed or reconfigured in different or alternative embodiments, and in other embodiments one or more method steps may be skipped altogether. Optional or desirable features of various device and system embodiments may be included in some embodiments and not in others. Therefore, the foregoing description is provided primarily for the purpose of example and should not be interpreted to limit the scope of the claims and specific embodiments or particular details or features disclosed.

As used herein in the specification and claims, including as used in the examples and unless otherwise expressly specified, all numbers may be read as if prefaced by the word “about” or “approximately,” even if the term does not expressly appear. The phrase “about” or “approximately” may be used when describing numeric values of magnitude and/or position to indicate that the value and/or position described is within a reasonable expected range of values and/or positions. For example, a numeric value may have a value that is +/−0.1% of the stated value (or range of values), +/−1% of the stated value (or range of values), +/−2% of the stated value (or range of values), +/−5% of the stated value (or range of values), +/−10% of the stated value (or range of values), etc. Any numerical values given herein should also be understood to include about or approximately that value, unless the context indicates otherwise.

For example, if the value “10” is disclosed, then “about 10” is also disclosed. Any numerical range recited herein is intended to include all sub-ranges subsumed therein. It is also understood that when a value is disclosed that “less than or equal to” the value, “greater than or equal to the value” and possible ranges between values are also disclosed, as appropriately understood by the skilled artisan. For example, if the value “X” is disclosed the “less than or equal to X” as well as “greater than or equal to X” (e.g., where X is a numerical value) is also disclosed. It is also understood that the throughout the application, data is provided in a number of different formats, and that this data, may represent endpoints or starting points, and ranges for any combination of the data points. For example, if a particular data point “10” and a particular data point “15” may be disclosed, it is understood that greater than, greater than or equal to, less than, less than or equal to, and equal to 10 and 15 may be considered disclosed as well as between 10 and 15. It is also understood that each unit between two particular units may be also disclosed. For example, if 10 and 15 may be disclosed, then 11, 12, 13, and 14 may be also disclosed.

What is claimed is:

1. A catheter control system, comprising:
a catheter control device comprising
a catheter port configured to receive the proximal end of a catheter, wherein the catheter comprises one or more pull wires coupled to a swashplate positioned at the proximal end of the catheter;
a steering device comprising
a flexible steering shaft configured to bend in response to a force being applied to the flexible steering shaft, and
a steering mechanism coupled to the steering shaft, the steering mechanism coupled to the swashplate when the catheter is coupled to the catheter control device, the steering mechanism configured to translate movement of the flexible steering shaft to movement of the swashplate to move a distal end of the catheter.

2. The catheter control system of claim 1, further comprising the catheter coupled to the catheter control device.

3. The catheter control system of claim 1, wherein the flexible steering shaft comprises one or more of an elastomer, a metallic spring, and a flexible metal tubing.

4. The catheter control system of claim 2, wherein the catheter control device is configured to move the distal end of the catheter up and down in a first plane.

5. The catheter control system of claim 4, wherein the catheter control device is configured to move the distal end of the catheter left and right in a second plane.

6. The catheter control system of claim 2, wherein the steering mechanism comprises
one or more pulleys; and
one or more steering wires disposed along the flexible steering shaft, routed through the one or more pulleys, and coupled to the one or more pull wires of the catheter via the swashplate,
wherein the one or more steering wires and the one or more pulleys are configured to, in response to the force being applied to the flexible steering shaft, transfer the force to the one or more pull wires of the catheter to move the distal end of the catheter.

7. The catheter control system of claim 1, wherein the steering mechanism is configured to move the distal end of the catheter in the same direction as the flexible steering shaft.

8. The catheter control system of claim 1, wherein the flexible steering shaft comprises two or more rigid members, and wherein the two or more rigid members are flexibly connected.

9. The catheter control device of claim 1, wherein the catheter port is further configured to receive the swashplate of the catheter and connect the one or more pull wires of the catheter to the one or more steering wires via the swashplate of the catheter.

10. The catheter control device of claim 1, wherein the flexible steering shaft comprises two or more rigid members, and wherein the two or more rigid members are flexibly connected.

11. A catheter control system, comprising:
a catheter control device, including
a catheter port configured to couple to a proximal end of a catheter that includes one or more pull wires;
a swashplate coupled to the one or more pull wires;
a steering shaft configured to bend in response to a force being applied to the steering shaft; and
a steering mechanism configured to translate the bend of the steering shaft to movement of the swashplate to correspondingly move the distal tip of a catheter coupled to the swashplate via the one or more pull wires.

12. The catheter control device of claim 11, wherein the steering mechanism includes a wire and at least one pulley configured to translate movement of the steering shaft to movement of the swashplate.

13. The catheter control device of claim 11, wherein the steering mechanism includes at least one hydraulic line.

14. The catheter control device of claim 11, wherein the steering mechanism a gear assembly.

15. The catheter control device of claim 11, wherein the steering mechanism is configured to move the swashplate to correspondingly move a distal tip of the catheter coupled to the swashplate in an anterior and posterior direction.

16. The catheter control device of claim 11, wherein the mechanism is further configured to move the swashplate to correspondingly move a distal tip of a catheter coupled to the swashplate in a left and right direction.

17. A catheter control system, comprising:
a catheter control device, including
a catheter port configured to receive the proximal end of a catheter having a swashplate, wherein the catheter comprises one or more pull wires;
a flexible steering shaft configured to bend in response to a force being applied to the flexible steering shaft;
a threaded member connected to the one or more pull wires via the catheter port;
a gear disposed along the threaded member such that the teeth of the gear interfaces with the threads of the threaded member; and
one or more steering wires disposed along the flexible steering shaft and connected to the gear,
wherein the one or more steering wires, the gear, and the threaded member are configured to, in response to the force being applied to the flexible steering shaft, transfer the force to the one or more pull wires of the catheter such that a distal end of the catheter moves in the same direction as the flexible steering shaft.

18. The catheter control device of claim 17, wherein the flexible steering shaft comprises one or more of an elastomer, a metallic spring, and a flexible metal tubing.

19. The catheter control device of claim 17, wherein the flexible steering shaft comprises two or more rigid members, and wherein the two or more rigid members are flexibly connected.

20. The catheter control device of claim 17, wherein the catheter port is further configured to receive a swashplate of the catheter and connect the one or more pull wires of the catheter to the threaded member via the swashplate of the catheter.
